SEQUENCE LISTING (26 sequences)

SEQ ID NO: 1 - protein, 107 aa, Artificial
  Synthesized 1FDL.asa light chain variable region
DIQMTQSPASLSASVGETVTITARASGNIHNYLAWYQQKQGKSPQLLVYYTTTLADGVPSRFSGSGSGTQYSLKINSLQPEDFGSYYAQHFWSTPRTFGGGTKLEIK

SEQ ID NO: 2 - protein, 107 aa, Artificial
  Synthesized 1FDL.rvp light chain variable region
DIQMTQSPASLSASVGETVTITCRASGNIHNYLAWYQQKQGKSPQLLVYYTTTLADGVPSRFSGSGSGTQYSLKINSLQPEDFGSYYCQHFWSTPRTFGGGTKLEIK

SEQ ID NO: 3 - protein, 115 aa, Artificial
  Synthesized 1FDL.asa heavy chain variable region
QVQLKESGPGLVAPSQSLSITDTVSGFSLTGYGVNWVRQPPGKGLEWLGMIWGDGNTDYNSALKSRLSISKDNSKSQVFLKMNSLHTDDTARYDARERDYRLDYWGQGTTLTVSS

SEQ ID NO: 4 - protein, 116 aa, Artificial
  Synthesized 1FDL.rvp heavy chain variable region
QVQLKESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTTLTVSS

SEQ ID NO: 5 - protein, 107 aa, Artificial
  Synthesized 1IGT.m light chain variable region
DIVLTQSPSSLSASLGDTITITCHASQNINVWLSWYQQKPGNIPKLLIYKASNLHTGVPSRFSGSGSGTGFTLTISSLQPEDIATYYCQQGQSYPLTFGGGTKLEIK

SEQ ID NO: 6 - protein, 107 aa, Artificial [sequence omitted]

SEQ ID NO: 7 - protein, 117 aa, Artificial
  Synthesized 1IGT.m heavy chain variable region
EVKLQESGGGLVQPGGSLKLSCATSGFTFSDYYMYWVRQTPEKRLEWVAYISNGGGSTYYPDTVKGRFTISRDNAKNTYLQMSRLKSEDTAMYYCARHGGYYAMDYWGQGTTVTVSS

SEQ ID NO: 8 - protein, 120 aa, Artificial [sequence omitted]

SEQ ID NO: 9 - protein, 101 aa, Artificial
  Synthesized 1IGT.m light chain constant domain
AKTTAPSVYPLAPVCGDTTGSSVTLGCYVKGYFIPEPVTLTWNSGSLSSGVHTFPAVILQSDLYTLSSSVTVTSSTWPSQSITCNVAHPASSTKVDKKIEP

SEQ ID NO: 10 - protein, 101 aa, Artificial
  Synthesized 1N8Z.h light chain constant domain
ASTKGPSVFPLAPSSKSTSGGTAALGCLVKDYTFPEPVTVSWNSGALTSGVHTFPAVLQSSGLYSLSSVVTVPSSSLGTQTYICNVNHKPSNTKVDKKVEP

SEQ ID NO: 11 - protein, 99 aa, Artificial
  Synthesized 1IGT.m heavy chain constant domain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEP

SEQ ID NO: 12 - protein, 100 aa, Artificial
  Synthesized 1N8Z.h heavy chain constant domain
ASTKGPSVFPLAPSSKSTSGGTAALGCLVKDYTTEPVTVSWNSGALTSGVHTFPAVLQSSGLYSLSSVVTVPSSSLGTQTYICNVNHKPSNTKVDKKVEP

SEQ ID NO: 13 - protein, 227 aa, Artificial
  Synthesized 1IGT.m Fc domain
RGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 14 - protein, 224 aa, Artificial
  Synthesized 1T83.h Fc domain
HTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 198 aa, Artificial
  Synthesized Reference kappa chain sequence
DIVLTQSPSSLSVSVGDRVTISCRASLAWYQQKPGKAPKLLIYSLESGVPSRFSGSGSGTDFTLTISSLQPEDFATYYCLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 213 aa, Artificial
  Synthesized Public kappa chain sequence
DVVMTQSPSSLSASVGDRVTITCQASQDISKYVNWYQQKPGRAPKLLIYEASNLETGVPPRFSGSGSGTHFSFTITGLQPEDIATYYCQQCDSLPPVFGQGTKLEVKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 196 aa, Artificial
  Synthesized Reference lambda chain sequence
QSVLTQPPSVSGSPGQTVTISCTGSVSWYQQKPGQAPKLVIYKRPSGIPERFSGSKSGNTASLTISGLQAEDEADYYCVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 216 aa, Artificial
  Synthesized Public lambda chain sequence
QSVLTQPPSVSAAPGQKVTISCSGSSSNIGKNYVSWYQQLPGAAPKLLIYESDKRPSGIPDRFSGSKSGTSATLGITGLQTGDEAAYYCGTWDHSLNAGVFGGGTTLTVLTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 406 aa, Artificial
  Synthesized Reference heavy chain sequence
QVQLVESGPELVKPSESLKLTCKVSVSWVRQAPGKGLEWVGVYYAPSVKGRVTISRDTSKNTAYLQLSSLRAEDTAVYYCWGSGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESKGQPENNYKTTPPVLDSDGSFFLYSKLTVDKSRWQQGNVFSCSVMHEALHNHYTQKSLSLSPGK

SEQ ID NO: 20 - protein, 453 aa, Artificial
  Synthesized Public heavy chain sequence
QVQLVQSGPEVKKPGSSVKVSCKASGGTFSSYAISWVRQAPGQGLEWMGGIIPIFGTANYAQKFQGRVTITADKSTSTAYMELSSLRSEDTAVYYCASTGYYDSSGYYYVPNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 406 aa, Artificial
  Synthesized Exemplary heavy chain sequence
QVQLVESGPELVKPSESLKLTCKVSVSWVRQAPGKGLEWVGVYYAPSVKGRVTISRDTSKNTAYLQLSSLRAEDTAVYYCWGSGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 451 aa, Artificial
  Synthesized Exemplary heavy chain sequence (GenBank Accession No. CAC10540)
EVQLLESGGGLVQPGGSLRLSCAASGFPFSNSAMSWVRQAPGKGLEWVSSISGSGGSTYYADSVKGRFTISRDNSKNTLSLQMNSLRAEDTAVYYCAKGGSSSGPYHFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 197 aa, Artificial
  Synthesized Exemplary kappa chain sequence
DIVLTQSPSSLSVSVGDRVTISCRASLAWYQQKGKAPKLLIYSLESGVPSRFSGSGSGTDFTLTISSLQPEDFATYYCLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 218 aa, Artificial
  Synthesized Exemplary kappa chain sequence (GenBank Accession No. BAC01559)
EIVLTQSPGTLSLSPGERATLSCRASQSVSSSYLAWYQQKPGQAPRLLIYGASSRATGIPDRFSGSGSGTDFTLTISRLEPEDSAVYYCQQYGSSPPWTFGQGTKLEIKRTVAAPSVFIFPFPSDEQLKSGTASVVCLLNNFYPREAKVQWKVDNALQSRGNSQESVTEQDSKDSTYSLSSTLTLSKADYEKHKVYACEVTHQGLSSPVTKSFNRGEC

SEQ ID NO: 25 - protein, 196 aa, Artificial; identical to SEQ ID NO: 17
  Synthesized Exemplary lambda light chain sequence

SEQ ID NO: 26 - protein, 213 aa, Artificial
  Synthesized Exemplary lambda light chain sequence (GenBank Accession No. CAE18238)
SVVTQEPSLTVSPGGTVTLTCGSSTGAVTSGHYPYWFQQKPGQAPRTLIYDVTNKDSWIPARFSGSLLGGKAALTLSGAQPEDEADYYCSLTYSGVRVFGGGTKLTVTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2011/0319598 A1
(43) Pub. Date: Dec. 29, 2011

(54) METHODS AND MATERIALS FOR DETERMINING ISOELECTRIC POINT

(75) Inventors: Mark Christopher Evans, Pleasant Hill, CA (US); Gary Studnicka, Santa Monica, CA (US)

(73) Assignee: XOMA TECHNOLOGY LTD., Berkeley, CA (US)

(21) Appl. No.: 13/140,554

(22) PCT Filed: Dec. 17, 2009
(86) PCT No.: PCT/US09/68531

§ 371 Date: Sep. 6, 2011

Related U.S. Application Data
(62) Division of application No. 61/138,408, filed on Dec. 17, 2008.
Application No. 61/138,408 is a division of application No. 61/138,411, filed on Dec. 17, 2008.

(51) Int. Cl. C07K 1/14; G06F 19/10

(52) U.S. Cl. 530/387.3; 530/412; 530/387.1; 702/19

(57) ABSTRACT

The present disclosure relates to methods and materials for determining an isoelectric point for a protein including, for example, a binding molecule such as an antibody. The isoelectric points may be used in methods for the preparation of proteins. Such methods may comprise identifying amino acid residues that are exposed on the surface of the protein in a sequence of amino acid residues of the protein, assigning a pKa value to the surface exposed amino acid residues, and calculating the isoelectric point of the protein from the pKa values assigned to the surface exposed amino acid residues. The methods of the present disclosure may be used for selecting and utilizing a buffer for purification of a protein, preparing a protein formulation, purifying a protein and/or stabilizing a protein in solution.

### CROSS-REFERENCE TO RELATED APPLICATIONS

This application claims priority to U.S. Provisional Application No. 61/138,408, filed on Dec. 17, 2008 and U.S. Provisional Application No. 61/138,411, filed on Dec. 17, 2008, each of which is hereby incorporated by reference in its entirety.

### FIELD

The present disclosure relates to methods and materials for determining an isoelectric point of a protein including, for example, a binding molecule such as an antibody. The isoelectric points may be used in methods for the preparation of proteins. The methods of the present disclosure may be used for selecting and utilizing a buffer for purification of a protein, preparing a protein formulation, purifying a protein and/or stabilizing a protein in solution.

### BACKGROUND

The isoelectric point (pL) of a molecule is the pH at which it has no net electrical charge. Biological molecules such as proteins are comprised of amino acids which may be positive, negative, neutral or polar in nature, and together give a protein its overall charge. At a pH below its pl, a protein carries a net positive charge while at a pH above its pl it carries a net negative charge. Lack of charge may have certain consequences on a protein. For example, proteins are often minimally soluble in water or buffers near their pl, which can lead to difficulties in the purification and/or formulation of therapeutics (Mosavi et al. (2003) Protein Engineering 16(10):739-745) and often precipitate out of solution.

The pl of a protein may be determined mathematically by several methods of calculation including, for example by using the Henderson-Hasselbalch equation. The pl of a protein may be computed by this equation by taking into account the acid-dissociation constant (pKa) of nine different chemical groups, including the side chains of seven amino acids, aspartic acid, glutamic acid, lysine, histidine, arginine, tyrosine and cysteine as well as the amino and carboxy terminal amino acid residues of the protein. Alternatively, the pl of a protein may be determined experimentally using isoelectic focusing. For example, when a protein is in a pH region below its isoelectric point (pl), it will be positively charged and so will migrate towards a cathode. As it migrates, however, the charge will decrease until the protein reaches the pH region that corresponds to its pl. At this point it has no net charge and so migration ceases. As a result, the proteins become focused into sharp stationary bands with each protein positioned at a point in the pH gradient corresponding to its pl. The technique is capable of extremely high resolution with proteins differing by a single charge being fractionated into separate bands. However, isoelectric focusing, although accurate in its determination of a protein's pl, may be time consuming and require laboratory resources making it not practical for widespread use. In contrast, pl values calculated mathematically can be determined quickly but may not make accurate predications of a protein's pl. Accordingly, improved methods are desired for the determination of a protein's pl that may have an accuracy more similar to isoelectric focusing but that are mathematically based.

### SUMMARY

The present disclosure relates to methods and materials for determining isoelectric points of proteins including, for example, binding molecules such as an antibodies. The isoelectric points may be used in methods for the preparation of proteins. The proteins may be prepared, for example, by identifying surface exposed amino acid residues in a sequence of amino acid residues of the protein; assigning a pKa value to the surface exposed amino acid residues; calculating the isoelectric point (pl) of the protein from the pKa values assigned to the surface exposed amino acid residues; preparing the protein by at least one of: selecting a buffer with a pH not equal to the calculated isoelectric point of the protein and utilizing the selected buffer for purification of the protein; and preparing a formulation of the protein with a pH not equal to the calculated isoelectric point of the protein.

The present disclosure provides methods for determining an isoelectric point of a protein by identifying surface exposed amino acid residues in a sequence of amino acid residues of the protein, assigning a pKa value to the surface exposed amino acid residues, and calculating the isoelectric point (pl) of the protein from the pKa values assigned to the surface exposed amino acid residues.

The present disclosure provides methods for selecting and utilizing a buffer for purification of a protein by identifying surface exposed amino acid residues in a sequence of amino acid residues of the protein, assigning a pKa value to the surface exposed amino acid residues, calculating an isoelectric point for the protein from the pKa values assigned to the surface exposed amino acid residues, selecting a buffer with a pH not equal to the calculated isoelectric point of the protein and utilizing the selected buffer for purification of the protein.

The present disclosure also provides methods of preparing a protein formulation by identifying amino acid residues that are exposed on the surface of the protein in a sequence of amino acid residues of the protein, assigning a pKa value to the surface exposed amino acid residues, calculating an isoelectric point for the protein from the pKa values assigned to the surface exposed amino acid residues, and preparing the formulation with a pH not equal to the calculated isoelectric point of the protein.

The present disclosure also provides method for purifying a protein from a heterogeneous population of proteins and/or other non-protein molecules and/or other contaminants by identifying amino acid residues that are exposed on the surface of the protein in a sequence of amino acid residues of the protein, assigning a pKa value to the surface exposed amino acid residues, calculating an isoelectric point (pl) for the protein from the pKa values assigned to the surface exposed amino acid residues, and utilizing the calculated pl to isolate the protein from the heterogeneous population of proteins.

The present disclosure also provides methods for stabilizing a protein in solution by identifying amino acid residues that are exposed on the surface of the protein in a sequence of amino acid residues of the protein, assigning a pKa value to the surface exposed amino acid residues, calculating an isoelectric point for the protein from the pKa values assigned to the surface exposed amino acid residues, preparing a formulation with a pH not equal to the calculated isoelectric point of the protein, and placing the protein in the prepared formulation.

The present disclosure also provides methods for determining an isoelectric point of a protein, the method comprising: receiving data indicative of a sequence of amino acid residues of the protein via an input device of a computing device; identifying surface exposed amino acid residues in the sequence of amino acid residues; assigning a pKa value to the surface exposed amino acid residues; calculating the isoelectric point (pl) of the protein from the pKa values assigned to the surface exposed amino acid residues using the computing device; and transferring the isoelectric point to an output device associated with the computing device.

The present disclosure also provides methods for determining an isoelectric point of an antibody, the method comprising: receiving data indicative of a sequence of amino acid residues of the antibody via an input device of a computing device; identifying surface exposed amino acid residues in the sequence of amino acid residues by aligning the sequence of amino acids residues of the antibody to a second antibody sequence of amino acid residues that are fixed to an IsoX line and are assigned an isoX value as shown in FIGS. 2-4 and wherein amino acid residues of the antibody have isoX values that are identical to corresponding positions in the second antibody; assigning a pKa value to the surface exposed amino acid residues; calculating the isoelectric point (pl) of the protein from the pKa values assigned to the surface exposed amino acid residues using the computing device; and transferring the isoelectric point to an output device associated with the computing device.

In some embodiments of any of the disclosed methods, the protein is a binding molecule such as an antibody or antibody fragment. In some embodiments of any of the disclosed methods, the antibody or antibody fragment is an IgG, a Fab or a scFv.

In some embodiments of any of the disclosed methods, the pKa values are assigned to the surface exposed amino acid residues by the system of EMBOSS, DTASelect, Solomon, Sillero, Rodwell, Patrickios or Wikipedia.

In some embodiments of any of the disclosed methods, all of the surface exposed amino acid residues are assigned a pKa value.

In some embodiments of any of the disclosed methods, the pl is calculated using the Henderson-Hasselbalch equation. In some embodiments of any of the disclosed methods, the pl is calculated using the method of EMBOSS, DTASelect, Solomon, Sillero, Rodwell, Patrickios or Wikipedia.

In some embodiments of any of the disclosed methods, the surface exposed amino acid residues are identified as those amino acid residues with an ASA value equal to or greater than 2. In some embodiments of any of the disclosed methods, the ASA values represent measured exposures for each amino acid residue. In some embodiments of any of the disclosed methods, the ASA values represent estimated exposures for each amino acid residue.

In some embodiments of any of the disclosed methods, the surface exposed amino acid residues are identified by aligning the sequence of amino acid residues of the antibody to a second antibody sequence of amino acid residues that are fixed to the “expo” line and are assigned an expo value as shown in FIGS. 1-4 and wherein amino acid residues of the antibody have expo values that are identical to corresponding positions in the second antibody. In some embodiments of any of the disclosed methods, the surface exposed amino acid residues are exposed, outward oriented (+) amino acid residues. In some embodiments of any of the disclosed methods, the surface exposed amino acid residues are partially exposed, surface oriented (o) amino acid residues. In some embodiments of any of the disclosed methods, the surface exposed amino acid residues are exposed, outward oriented (+) amino acid residues and partially exposed, surface oriented (o) amino acid residues.

In some embodiments of any of the disclosed methods, the surface exposed amino acid residues are identified by aligning the sequence of amino acids residues of the antibody to a second antibody sequence of amino acid residues that are fixed to an IsoX line and are assigned an isoX value as shown in FIGS. 2-4 and wherein amino acid residues of the antibody have isoX values that are identical to corresponding positions in the second antibody. In some embodiments, the surface exposed amino acid residues are “•” amino acid residues.

In some embodiments of any of the disclosed methods, the buffer/formulation is used for pharmaceutical administration.

In some embodiments of any of the disclosed methods, the pH of the selected buffer/formulation is greater than the pl of the protein. In some embodiments of any of the disclosed methods, the pH of the selected buffer/formulation is less than the pl of the protein.

Additional features and advantages are described herein, and will be apparent from, the following Detailed Description and the Figures.

### BRIEF DESCRIPTION OF THE FIGURES

FIG. 1A-B shows the alignment of the light and heavy chain variable domain of 1FDL on an “expo” line. Also indicated are ASA values representing exposure determined from the crystal structure of 1FDL (.asa) and ASA values representing an estimate of exposure (.rvp). Highlighted amino acid residues indicate positions at which cysteine (C) residues are incorrectly indicated as another amino acid residue.

FIG. 2A-B show an alignment of the light and heavy chain variable domain of an exemplary murine antibody (1 IGT.m) and human antibody (1N8Z.h) on an “expo” line and an “IsoX” line. Further, ASA values are indicated for amino acid residues in both the murine and human antibody.

FIG. 3 shows an alignment of the light and heavy chain constant region of an exemplary murine antibody (1 IGT.m) and human antibody (1N8Z.h) on an “expo” line and an “IsoX” line. Further, ASA values are indicated for amino acid residues in both the murine and human antibody.

FIG. 4A-B show an alignment of the Fc domain of an exemplary murine antibody (1IGT.m) and human antibody (1N8Z.h) on an “expo” line and an “IsoX” line. Further, ASA values are indicated for amino acid residues in both the murine and human antibody.

FIG. 5 is a flowchart showing one example of a process for displaying an isoelectric point associated with an amino acid sequence of an antibody.

FIG. 6 is a screen shot of an example user interface for displaying alphabetic strings indicative of a light chain.

FIG. 7 is another screen shot of an example user interface for displaying alphabetic strings indicative of a light chain.

FIG. 8 is another screen shot of an example user interface for displaying alphabetic strings indicative of a heavy chain.

FIG. 9 shows an exemplary heavy chain (Genbank Accession No. CAC10540) amino acid sequence (second row of amino acid sequences) aligned with a second amino acid sequence (first row of amino acid sequence).

FIG. 10 shows an exemplary kappa light chain (Genbank Accession No. BAC01559) amino acid sequence (second row of amino acid sequences) aligned with a second amino acid sequence (first row of amino acid sequence).

FIG. 11 shown an exemplary lambda light chain (Genbank Accession No. CAE18238) amino acid sequence (second row of amino acid sequences) aligned with a second amino acid sequence (first row of amino acid sequence).

### DETAILED DESCRIPTION

The present disclosure provides methods and materials for determining an isoelectric point for a protein including, for example, a binding molecule such as an antibody (e.g., an IgG, a Fab or a scFv). An isoelectric point determined by any of the disclosed methods or materials may be used in methods to prepare a protein, including an antibody. The protein may be prepared, for example, by identifying surface exposed amino acid residues in a sequence of amino acid residues of the protein; assigning a pKa value to the surface exposed amino acid residues; calculating the isoelectric point (pl) of the protein from the pKa values assigned to the surface exposed amino acid residues; preparing the protein by at least one of: selecting a buffer with a pH not equal to the calculated isoelectric point of the protein and utilizing the selected buffer for purification of the protein; and preparing a formulation of the protein with a pH not equal to the calculated isoelectric point of the protein. Surprisingly, it has been found that the pl of a protein calculated from amino acid residues located on the surface of a protein (referred to herein as “surface exposed amino acid residues) approaches the pl of the protein as determined by isoelectric focusing. Such methods may be used to determine the isoelectric point of a protein, select and utilize a buffer for purification of a protein, prepare a protein formulation, purify a protein from a heterogeneous population of proteins and/or stabilize a protein in solution.

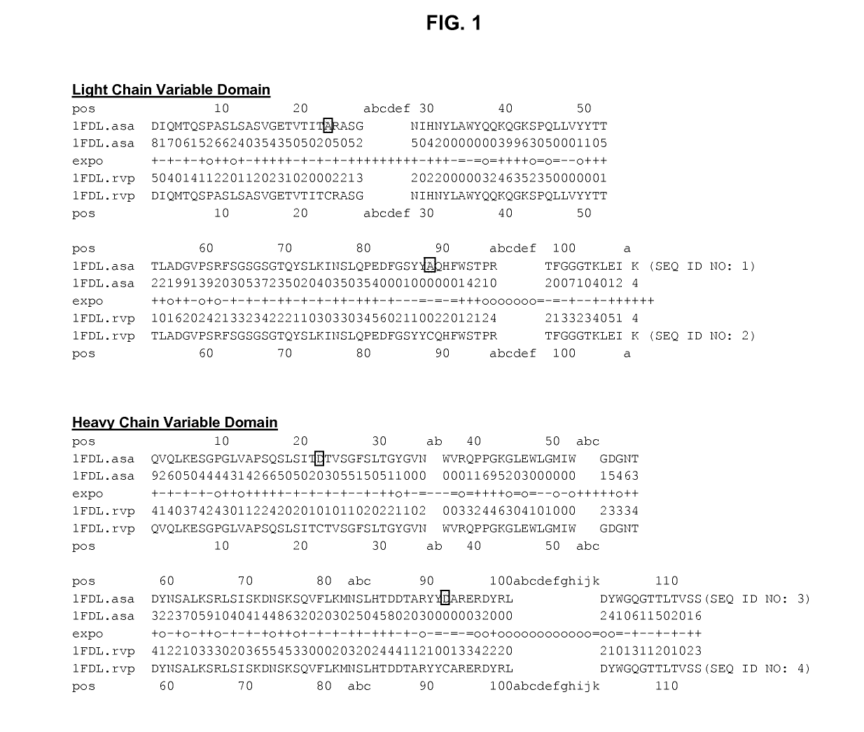

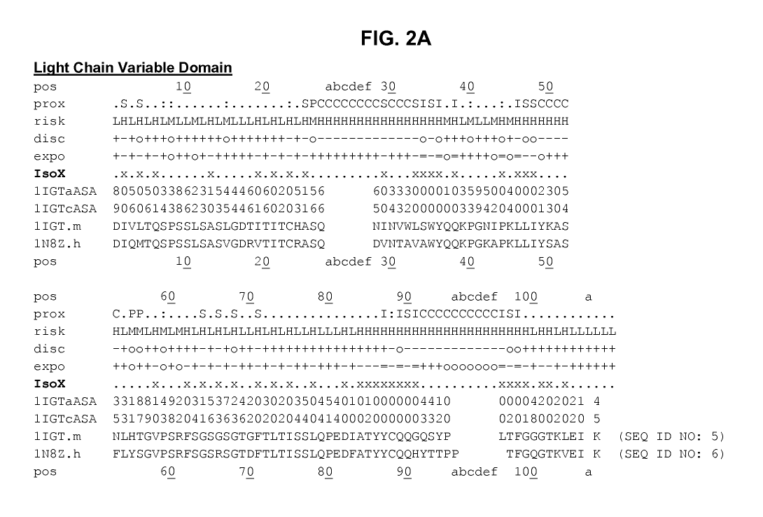

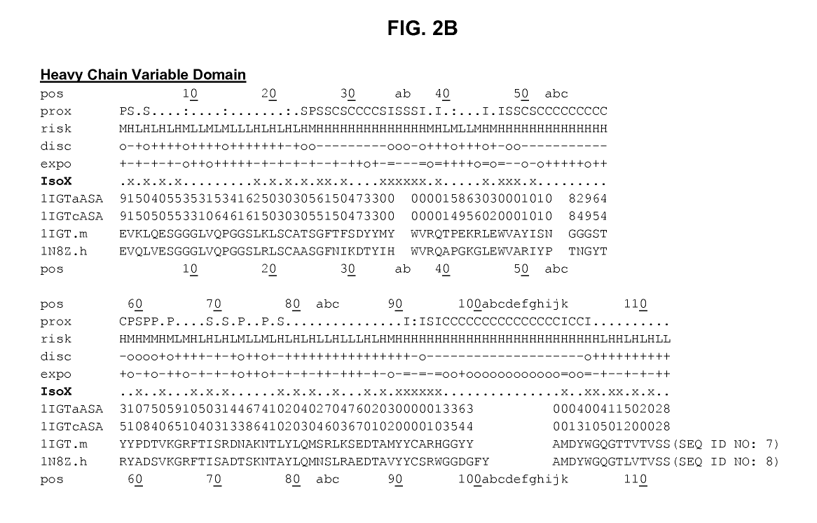

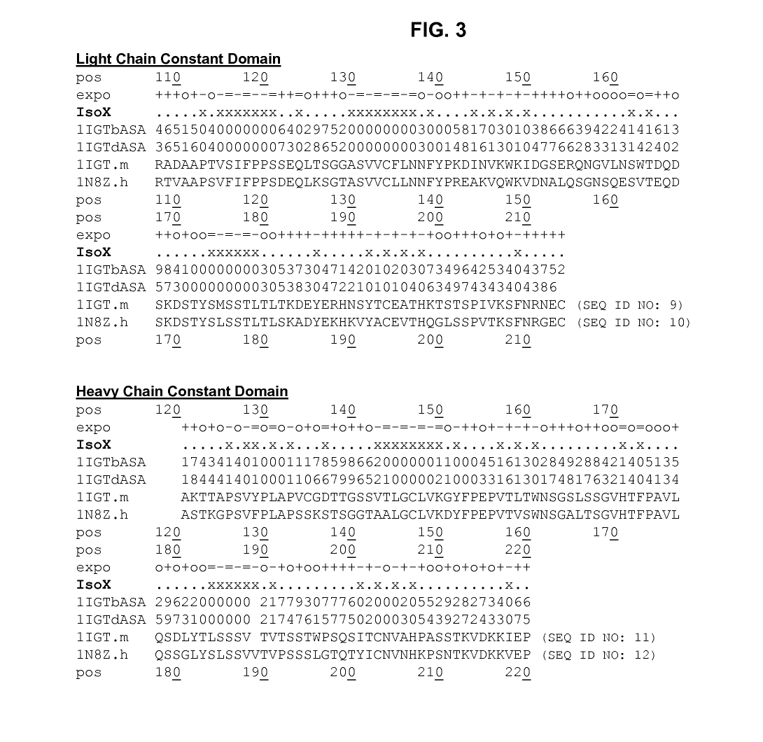

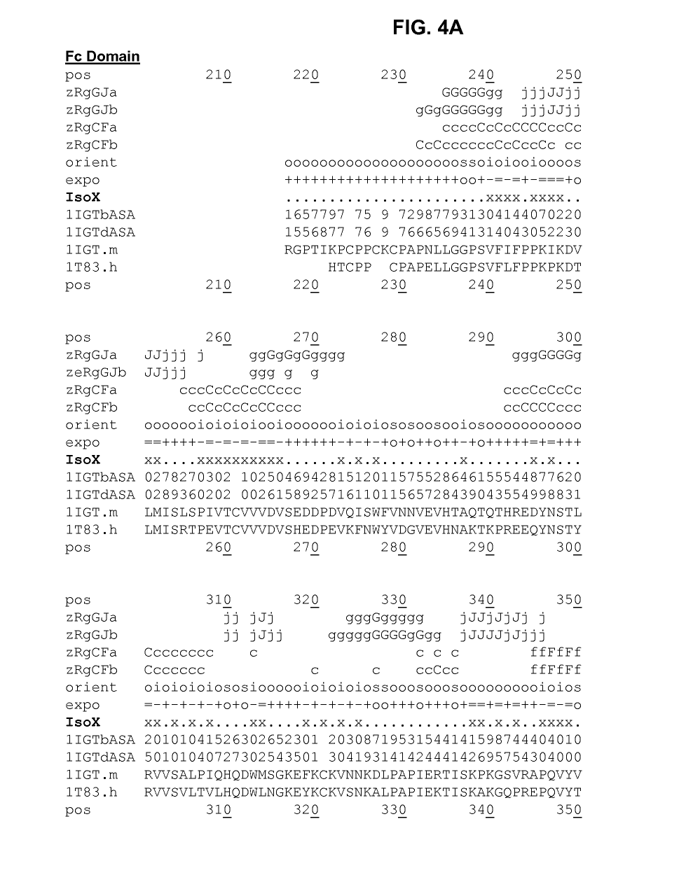

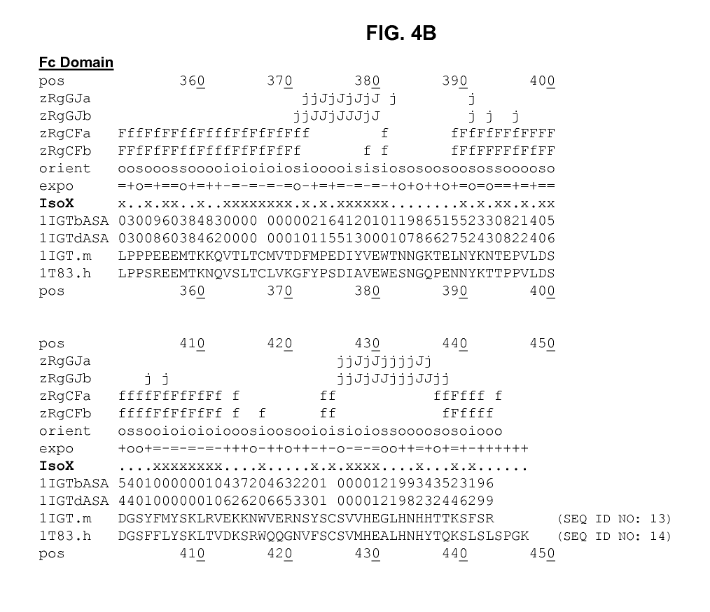

Methods provided by the present disclosure may be used for determining an isoelectric point of protein including, for example, a binding molecule such as an antibody or binding fragment thereof by identifying amino acid residues that are surface exposed, assigning a pKa value to the surface exposed amino acid residues, and calculating the isoelectric point of the protein from the pKa values assigned to the surface exposed amino acid residues and optionally but preferably the pKa values assigned to amino and carboxy terminal amino acid residues. Amino acid residues that are surface exposed may be identified by determining their ASA value. Alternatively, amino acid residues that are surface exposed may be identified using the “expo” line as shown in FIGS. 1-4 or by using the “IsoX” line as shown in FIGS. 2-4. Selected amino acid residues including, for example, cysteine (Cys, C), aspartic acid (Asp, D), glutamic acid (Glu, E), histidine (H is, H), lysine (Lys, K), arginine (Arg, R) and/or tyrosine (Tyr, Y) that are identified as exposed on the surface of a protein may be used to calculate surface pl of the protein.

The present disclosure also provides methods for determining an isoelectric point of an antibody by identifying amino acid residues in the antibody with an ASA value equal to or greater than 2 as surface exposed, assigning a pKa value to the surface exposed amino acid residues, and calculating the isoelectric point (pl) of the antibody from the pKa values assigned to the surface exposed amino acid residues.

The present disclosure also provides methods for determining an isoelectric point of an antibody or binding fragment thereof by aligning the sequence of amino acid residues of the antibody to a second antibody sequence of amino acid residues that are fixed to the “expo” line and are assigned an expo value as shown in FIGS. 1-4, wherein amino acid residues of the antibody have expo values that are identical to corresponding positions in the second antibody and wherein, outward oriented (+) amino acid residues and/or partially exposed, surface oriented (o) amino acid residues are identified as surface exposed, assigning a pKa value to the surface exposed amino acid residues, and calculating the isoelectric point (pl) of the protein from the pKa values assigned to the surface exposed amino acid residues.

The present disclosure also provides methods for determining an isoelectric point of an antibody or binding fragment thereof by aligning the sequence of amino acids residues of the antibody to a second antibody sequence of amino acid residues that are fixed to an IsoX line and are assigned an isoX value as shown in FIGS. 2-4, wherein amino acid residues of the antibody have isoX values that are identical to corresponding positions in the second antibody and wherein “•” amino acid residues are identified as surface exposed, assigning a pKa value to the surface exposed amino acid residues, and calculating the isoelectric point (pl) of the protein from the pKa values assigned to the surface exposed amino acid residues.

The present disclosure provides methods for selecting and utilizing a buffer for purification of a protein including, for example, a binding molecule such as an antibody by identifying surface exposed amino acid residues in a sequence of amino acid residues of the protein, assigning a pKa value to the surface exposed amino acid residues, calculating an isoelectric point for the protein from the pKa values assigned to the surface exposed amino acid residues, selecting a buffer with a pH not equal to the calculated isoelectric point of the protein and utilizing the selected buffer for purification of the protein.

The present disclosure also provides methods of preparing a protein including, for example, a binding molecule such as an antibody formulation by identifying amino acid residues that are exposed on the surface of the protein in a sequence of amino acid residues of the protein, assigning a pKa value to the surface exposed amino acid residues, calculating an isoelectric point for the protein from the pKa values assigned to the surface exposed amino acid residues, and preparing the formulation with a pH not equal to the calculated isoelectric point of the protein.

The present disclosure also provides method for purifying a protein including, for example, a binding molecule such as an antibody from a heterogeneous population of proteins and/or other non-protein molecules and/or other contaminants by identifying amino acid residues that are exposed on the surface of the protein in a sequence of amino acid residues of the protein, assigning a pKa value to the surface exposed amino acid residues, calculating an isoelectric point (pl) for the protein from the pKa values assigned to the surface exposed amino acid residues, and utilizing the calculated pl to isolate the protein from the heterogeneous population of proteins.

The present disclosure also provides methods for stabilizing a protein including, for example, a binding molecule such as an antibody in solution by identifying amino acid residues that are exposed on the surface of the protein in a sequence of amino acid residues of the protein, assigning a pKa value to the surface exposed amino acid residues, calculating an isoelectric point for the protein from the pKa values assigned to the surface exposed amino acid residues, preparing a formulation with a pH not equal to the calculated isoelectric point of the protein, and placing the protein in the prepared formulation.

The present disclosure also provides methods for determining an isoelectric point of a protein including, for example, a binding molecule such as an antibody, the method comprising: receiving data indicative of a sequence of amino acid residues of the protein via an input device of a computing device; identifying surface exposed amino acid residues in the sequence of amino acid residues; assigning a pKa value to the surface exposed amino acid residues; calculating the isoelectric point (pl) of the protein from the pKa values assigned to the surface exposed amino acid residues using the computing device; and transferring the isoelectric point to an output device associated with the computing device.

The present disclosure also provides methods for determining an isoelectric point of an antibody, the method comprising: receiving data indicative of a sequence of amino acid residues of the antibody via an input device of a computing device; identifying surface exposed amino acid residues in the sequence of amino acid residues by aligning the sequence of amino acids residues of the antibody to a second antibody sequence of amino acid residues that are fixed to an IsoX line and are assigned an isoX value as shown in FIGS. 2-4 and wherein amino acid residues of the antibody have isoX values that are identical to corresponding positions in the second antibody; assigning a pKa value to the surface exposed amino acid residues; calculating the isoelectric point (pl) of the protein from the pKa values assigned to the surface exposed amino acid residues using the computing device; and transferring the isoelectric point to an output device associated with the computing device.

In referring to a pH “not equal to” the calculated isoelectric point, the present disclosure contemplates that a range of pH values may be utilized which differ (e.g., greater than, less than) from the calculated isoelectric point. For example, a pH “not equal to” the calculated isoelectric point may represent a numerical difference in pH values (e.g., 6.5 versus 6.0), a functional difference in protein solubility (e.g., when selecting a buffer for purification of a protein and/or preparing a formulation of a protein), or preferably both. Preferably, the pH should differ from (e.g., not equal to) the calculated isoelectric point, so as to reduce or prevent aggregation or precipitation of the protein, such as for example in selecting a buffer for purification of the protein and/or preparing a formulation of the protein.

In some embodiments, the pH may be at least about 0.2 pH units, at least about 0.3 pH units, at least about 0.4 pH units, at least about 0.5 pH units, at least about 0.6 pH units, at least about 0.7 pH units, at least about 0.8 pH units, at least about 0.9 pH units, at least about 1.0 pH units, at least about 1.2 pH units, at least about 1.5 pH units, or at least about 2.0 pH units greater than or less than the calculated isoelectric point as disclosed herein. Alternatively or in addition, in some embodiments, the pH may be at least about 2%, at least about 3%, at least about 4%, at least about 5%, at least about 6%, at least about 7%, at least about 8%, at least about 9%, at least about 10%, at least about 12%, at least about 15%, or at least about 20% greater than or less than the calculated isoelectric point as disclosed herein.

### Identification of Surface Exposed Residues

The present disclosure provides novel methods for identifying one or more surface exposed amino acid residues including, for example, each surface exposed amino acid residue in a sequence of amino acid residues from a protein of interest (e.g., an antibody or binding fragment thereof, such as an IgG, Fab or scFv). Surface exposed amino acid residues may be identified by their ASA value, by using the “expo” line as shown in FIGS. 1-4 or by using the “IsoX” line as shown in FIGS. 2-4.

An ASA value for each amino acid position in a protein may be used to identify those amino acid residues that are surface exposed (see, e.g., http://www.netasa.org/asaview/, referred to herein as “Netasa web server” and Ahmad et al. (2004) BMC Bioinformatics 5:51). Surface exposed amino acid residues may be identified as those amino acid residues with an ASA value equal to or greater than 2. ASA values for a protein may be viewed in the form of a bar graph as shown by the Netasa web server, in which a linear amino-acid sequence may be plotted along the horizontal axis, and the degree of solvent exposure for each residue represented by the height of a vertical bar, whose color-coding distinguishes the sidechain as nonpolar (e.g., grey) or polar (e.g., green) or negative (e.g., red) or positive (e.g., blue) or cysteine (e.g., yellow). These bar graphs may depict groups of exposed (e.g., tall bar) or buried (e.g., short bar) amino acid residues, as well as the linear distribution of polarity and charge. Additionally or alternatively, ASA values for a protein can be obtained in numerical form as a text-only file and exported to programs that allow manipulation of the data (e.g., Microsoft Word or Excel). ASA values may be represented as single digit (from “0” to “9”), corresponding to the “tens digit” of the exposure percentage (ranging from 0% to 100% exposed). Thus, for example, 37.1% exposure is coded as “3”, while 52.7% is coded as “5”. Note that 4.6% is coded as “0”, since it represents 04.6%. Also, to preserve the single-digit scheme, 100.0% is coded as “9”, since it is nearly equivalent to 99.9%.

When the crystal structure of the protein is known ASA values represent measured exposures for each amino acid residue (see, e.g., Ahmad et al. (2002) Bioinformatics 18:819-824). ASA values obtained from a crystal protein structure are obtained as “.asa” files (see, e.g., Table 1) from the Netasa web server and may be represented on a text line. Text lines may display information including, for example, information about the surface exposure of an amino acid residue in a protein. For example, on a text line such as “E83 27.2”, (“E”) is the one-letter amino-acid code for glutamic acid, (“83”) is the non-Kabat position number in the linear sequence and (“27.2”) is the ASA coefficient of surface exposure to solvent percent exposure of the residue's total surface area.

Alternatively, when a protein's crystal structure is not known, ASA values may represent estimated exposures for each amino acid residue based upon the statistical frequencies of various linear amino-acid fragments among a large group of crystallized proteins (Ahmad et al. (2003) Bioinformatics 19:1849-1851). ASA values obtained from a protein in which the crystal structure is unknown are obtained as “.rvp” files (see, e.g., Table 1) from the Netasa web server and may be represented on a text line. Text lines may display information including, for example, information about the surface exposure of an amino acid residue in a protein. For example, on a text line such as “83 E 27.2 47.6 E”, (“83”) is the non-Kabat position number in the linear protein sequence, (“E”) is the one-letter amino-acid code for glutamic acid, (“27.2”) is the ASA (RVP) statistical estimate of surface exposure to solvent, (“47.6”) is the AA2 value in square angstroms of the amount of exposed surface area and (“E”) is the one-letter category-designation for buried or exposed, based on a threshold percentage.

In certain cases, ASA view may provide an incorrect one letter amino acid code at a position in protein. For example, cysteine residues in the variable domain of an antibody may be represented as an amino acid other than a (C). Also, in some instances ASA view inserts amino acids (e.g., using two letter codes) at various amino acid positions in the protein sequence. Accordingly, it may be useful to manually edit the ASA test file before processing.

ASA values for proteins in which the complete three-dimensional structure is known may be calculated using programs such as ACCESS, DSSP, ASC, NACCESS, or GETAREA. Furthermore, the ASA values can also be obtained directly from the DSSP database, if the corresponding PDB code is known.

Surface exposed amino acid residues may also be identified by using the asa line as shown in FIGS. 1-4. In an exemplary method, surface exposed amino acid residues in an antibody of binding fragment thereof may be identified by aligning the amino acid sequence of the antibody to a second sequence of amino acids fixed to the asa line of FIGS. 1-4, wherein amino acid residues of the antibody have asa values that are identical to corresponding positions in the second antibody fixed to the asa line. Amino acid residues that have an ASA value greater than or equal to 2 may be identified as surface exposed.

In those instances when a protein's crystal structure is not known, surface exposed amino acid residues may be determined by using the “expo” line of FIGS. 1-4. In an exemplary method, surface exposed amino acid residues in an antibody or binding fragment thereof may be identified by aligning the amino acid sequence of the antibody to a second sequence of amino acids fixed to the “expo” line of FIGS. 1-4, wherein amino acid residues of the antibody have “expo” values that are identical to corresponding positions in the second antibody fixed to the “expo” line. The “expo” line classifies the surface exposure of each amino-acid position into one of the four following categories: “+” (exposed, outward oriented) or “o” (partially exposed, surface oriented) or “−” (buried in core, inward oriented) or “=” (buried in interface, inward oriented) (Studnicka et al. (1994) Protein Eng. 7(6):805-814; U.S. Pat. No. 5,766,886). Surface exposed amino acid residues may be those amino acid residues that are classified as exposed, outward oriented (+) amino acid residues and/or those amino acid residues that are classified as partially exposed, surface oriented (o) amino acid residues.

In another exemplary method, when the crystal structure of a protein, is unknown, surface exposed amino acid residues in the protein may be identified by using the “IsoX” line as shown in FIGS. 2-4. In an exemplary method, surface exposed amino acid residues in an antibody or binding fragment thereof may be identified by aligning the amino acid sequence of the antibody to a second sequence of amino acids fixed to the “IsoX” line of FIGS. 1-3, wherein amino acid residues of the antibody have “IsoX” values that are identical to corresponding positions in the second antibody fixed to the “IsoX” line. The “IsoX” line categorizes each amino acid residue as surface exposed (“•”) or buried (“x”). Amino acid residues from the protein of interest that match a corresponding residue in the sequence fixed to the “IsoX” line may be assigned the same “IsoX” value that is assigned to the amino acid residue in the fixed sequence. Amino acid residues from the protein of interest that do not have a corresponding match with the fixed sequence may be considered as surface exposed “•”. Alternatively, amino acid residues from the protein of interest that do not have a corresponding match with the fixed sequence may be considered as buried “x”. The amino acid sequence fixed to the “IsoX” line may be any selected antibody sequence including, for example, an antibody germline sequence or antibody consensus sequence. Antibody sequences aligned and/or fixed to the “IsoX” line may comprise light and/or heavy chain variable regions and/or light and/or heavy chain constant regions.

Non-conserved amino acid residues in an antibody or binding fragment of interest including, for example, complementarity determining regions (CDRs) or mutations, that do not match a corresponding residue in the sequence of amino acid residues fixed to the “IsoX” line may be considered as surface exposed. Alternatively, non-conserved amino acid residues in an antibody of binding fragment including, for example, complementarity determining regions (CDRs) or mutations, that do not match a corresponding residue in the sequence fixed to the IsoX line may be considered as buried. In some embodiments, amino acid residues from the antibody or binding fragment of interest that are in the CDRs and do not match a corresponding residue in the sequence fixed to the “IsoX” line may be considered as surface exposed while all other amino acid residue mismatches are considered as buried residues.

Without wishing to be bound by a theory of the invention, it is believed that the identification of surface exposed amino acid residues from the “IsoX” line are likely to be more precise than the ASA statistical estimates because they represent the conserved structural features of antibody molecules. However, when an antibody's crystal structure is known, the ASA-View coefficients may be more precise than the average conserved exposures represented by the “IsoX” line.

Moreover, surface exposed amino acid residues may be identified by using tables based on short peptide fragments (e.g., 3 to 5 amino acids in length) from proteins with known and well-characterized crystal structures (Ahmad et al. (2003) Genome Informatics 14:482-483). Table entries may contain the statistical frequencies of exposure or burial for the middle residue (“X”) in each short fragment (O-X-O or O-O-X-O-O), as a function of its close neighbors (“O”) on either side. Additionally, Fourier transform mass spectrometry may be employed to detect the reactivity of side-chain groups to chemical modification, such as acetylation of primary amines (Novak et al. (2004) J. Mass Spectrom. 39:322-328). Side chain that are more reactive to chemical modification may be indicated as exposed.

### Methods for Calculating an Isoelectric Point of a Protein

The isoelectric point of a protein, for example, an antibody such as an scFv may be calculated mathematically by using acid-dissociation constant (“pKa”) values assigned to certain individual amino acid residues.

In an exemplary method an isoelectric point for a protein may be determined by using nine different chemical groups, including the sidechains of seven amino acids and their amino and carboxy termini. These amino acids may include: cysteine (Cys, C), aspartic acid (Asp, D), glutamic acid (Glu, E), histidine (H is, H), lysine (Lys, K), arginine (Arg, R) and tyrosine (Tyr, Y). The pl of a protein may be computed using the Henderson-Hasselbalch equation which takes into account the logarithm of the pKa for each of the nine chemical groups. In a protein, each of the nine chemical groups may be present in zero or more copies (“N”) per molecule, all of which contribute proportionally to the final pl. Thus, for example, the Henderson-Hasselbalch contribution of lysine must be multiplied by N_{K}=7 in a protein containing seven lysines.

An exemplary algorithm utilizes a formula for the total concentration of charges associated with each amino acid, both for anionic [A⁻] species (e.g., D, E, Y, C, or the carboxy terminus) and for cationic [HA⁺] species (e.g., K, H, R, or the amino terminus). The mathematical basis for algorithms for calculation of pl involves converting the Henderson-Hasselbalch equation from logarithmic to exponential form as shown below:

pKa=pH+log([HA]/[A⁻])

pKa=pH+log([HA⁺]/[A])

pKa=pH+log([HA]/[A⁻])

pKa=pH−log([A]/[HA⁺])

pKa=pH+log([HA]/[A⁻])

−pKa=−pH+log([A]/[HA⁺])

10̂(pKa−pH)=([HA]/[A⁻])

10̂(pH−pKa)=([A]/[HA⁺])

1+(10̂(pKa−pH))=1+[HA]/[A⁻])

1+(10̂(pH−pKa))=1+[A]/[HA⁺])

1+(10̂(pKa−pH))=(([HA]+[A])/[A])

1+(10̂(pH−pKa))=(([A]+[HA⁺])/[HA⁺])

Next, a separate equation may be set out for the total charge C contributed by N copies of each positive or negative amino-acid species:

C=−N[A⁻]/([HA]+[A⁻])

C=+N [HA⁺]/([A]+[HA⁺])

Rearranging this gives:

(([HA]+[A⁻])/[A⁻])=((−N)/C)

(([A]+[HA⁺])/[HA⁺])=((+N)/C)

Substituting this into the Henderson-Hasselbalch equation eliminates the references to concentrations:

1+(10̂(pKa−pH))=((−N)/C)

1+(10̂(pH−pKa))=((+N)/C)

Solving for the charge C gives:

C=−N/(1+(10̂(pKa−pH)))

C=N/(1+(10̂(pH−pKa)))

Finally, nine separate versions of these two equations are generated, each with a different chemical group represented by the subscript “i”—either anionic (e.g., D, E, Y, C, or carboxy) in the top equation, or cationic (e.g., K, H, R, or amino) in the bottom equation:

Cᵢ=−Nᵢ/(1+(10̂(pKaᵢ−pH)))

Cᵢ=Nᵢ/(1+(10̂(pH−pKaᵢ)))

The total charge T contributed by all nine species is:

T=C_{D}+C_{E}+C_{K}+C_{H}+C_{R}+C_{Y}+C_{C}+Cₐₘᵢₙₒ+C_{carboxy}

The sum T of all charges from all the different amino-acid species equals zero at the isoelectric point, which may be somewhere between pH 0 and pH 14. To begin the iterative process, a trial pH may be chosen in the middle at pH 7, and this value then plugged into the equation to determine whether the total charge T is positive or negative or zero at the trial pH. On the one hand, if this charge T is positive, then the pl must be greater than the trial pH. It must lie between the trial value (pH 7) and the highest untested value (pH 14), so a new trial pH is chosen in the middle at pH 10.5. On the other hand, if this charge T is negative, then the pl must be less than the trial pH. It must lie between the lowest untested value (pH 0) the and trial value (pH 7), so a new trial pH is chosen in the middle at pH 3.5. Each time this “binary search” cycle is repeated, the remaining range of possible untested pl values will be cut in half (or “bisected”), and the calculation will quickly converge to the correct pl value, when the total charge T finally becomes zero.

Computer programs may be employed to determine the pl of a protein (see, e.g., Sillero et al. (2006) Comput Biol Med. 36(2):157-66; Hennig (2001) Prep Biochem Biotechnol. 31(2):201-207; Ribeiro et al. (1991) Comput Biol Med. 21(3):131-141; Ribeiro et al. (1990) Comput Biol Med. 20(4):235-42; Tabb's DTASelect algorithm at “http://fields.scripps.edu/DTASelect/20010710-pl-Algorithm.pdf; and the QT4 version of the isoelectric point calculator at “http://isoelectric.ovh.org/files/isoelectric-point-windows.zip). Although most algorithms consider the protonation or deprotonation of each ionizable residue in isolation, others may account for the influence of the local chemical environment generated by neighboring residues in the primary sequence. For example, one method based on a 5000-peptide database takes into account the effect of adjacent amino acids on the pl value (see, e.g., Cargile et al. (2008) Electrophoresis 29(13):2768-2778.

Minor variations of the algorithm derived above include, for example, EMBOSS, DTASelect, Solomon, Sillero, Rodwell, Patrikios or Wikipedia. Such methods accept the linear amino-acid sequence of a protein, without utilizing any additional structural information (e.g., surface exposure) to direct their calculations. However, they disagree about the pKa values associated with the various amino acids and termini. PKa values assigned to the nine chemical groups by these methods are shown in Table 1.

TABLE 1
PKa Values Associated with Various Amino Acids and Their Termini

| | C | D | E | H | K | R | Y | NH₂ | COOH |
|---|---|---|---|---|---|---|---|---|---|
| EMBOSS | 8.5 | 3.9 | 4.1 | 6.5 | 10.8 | 12.5 | 10.1 | 8.6 | 3.6 |
| DTASelect | 8.5 | 4.4 | 4.4 | 6.5 | 10.0 | 12.0 | 10.0 | 8.0 | 3.1 |
| Solomon | 8.3 | 3.9 | 4.3 | 6.0 | 10.5 | 12.5 | 10.1 | 9.6 | 2.4 |
| Sillero | 9.0 | 4.0 | 4.5 | 6.4 | 10.4 | 12.0 | 10.0 | 8.2 | 3.2 |
| Rodwell | 8.33 | 3.68 | 4.25 | 6.0 | 11.5 | 11.5 | 10.07 | 8.0 | 3.1 |
| Patrickios | — | 4.2 | 4.2 | — | 11.2 | 11.2 | — | 11.2 | 4.2 |
| Wikipedia | 8.18 | 3.9 | 4.07 | 6.04 | 10.54 | 12.48 | 10.46 | 8.2 | 3.65 |

Methods for assigning a pKa value to an amino acid residue may take into account the interaction between a particular residue and the local environment created by surrounding residues. For example, pKa values may be assigned to amino acid residues based on experimental pKa values determined in protein chains with known structures (He, et al. (2007) Proteins 69(1):75-82). Other methods for calculating the pKa values of ionizable groups in proteins may be based on a distance and position dependent screening of the electrostatic potential (see, e.g., Sandberg et al. (1999) Proteins 36(4):474-483). Additionally, methods based on experimental isoelectric points and amino acid compositional data may uses linear regression to estimate pKa values for ionizable alpha and beta positions of acidic or basic amino-acid residues (Patrickios et al. (1995) Anal Biochem. 231(1):82-91).

### Methods for Displaying an Isoelectric Point of a Protein

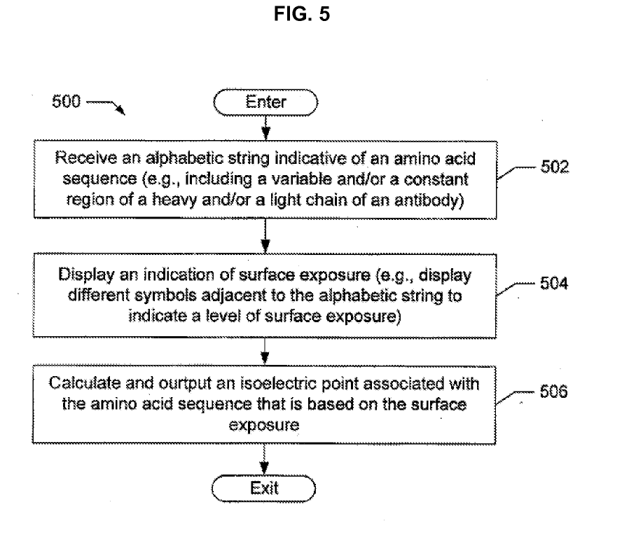

A flowchart of an example process 500 for displaying an isoelectric point associated with an amino acid sequence of an antibody is presented in FIG. 5. Preferably, the process 500 is embodied in one or more software programs which are stored in one or more memories and executed by one or more processors of a computing device, some of the steps described may be optional, and additional steps may be included.

A computing device begins the example process 500 by receiving an alphabetic string indicative of an amino acid sequence (block 502). For example, a user may enter the alphabetic string using an input device such as a keyboard, or the user may retrieve the alphabetic string from a database, such as a database stored on the computing device or a network device (e.g., the IMGT germ line sequence database, the Kabat database, etc.). The amino acid sequence represented by the alphabetic string may include a variable region and/or a constant region of a heavy chain and/or a light chain of an antibody (e.g., an antibody or fragment thereof such as an IgG, a Fab or a scFv). In some embodiments, the alphabetic string may include a partial or full-length heavy and/or light chain of an antibody. In some embodiments, the alphabetic string may include a variable region of a heavy and/or light chain of an antibody. In some embodiments, the alphabetic string may include a variable region of a heavy chain and/or one or more constant regions of a heavy chain (e.g., C_{H}1, C_{H}2 and/or C_{H}3) and/or a variable region of a light chain and/or a constant region of a light chain (e.g., C_{L}) of an antibody. In some embodiments, the alphabetic string may include two full-length heavy chains and/or two full-length light chains of an antibody.

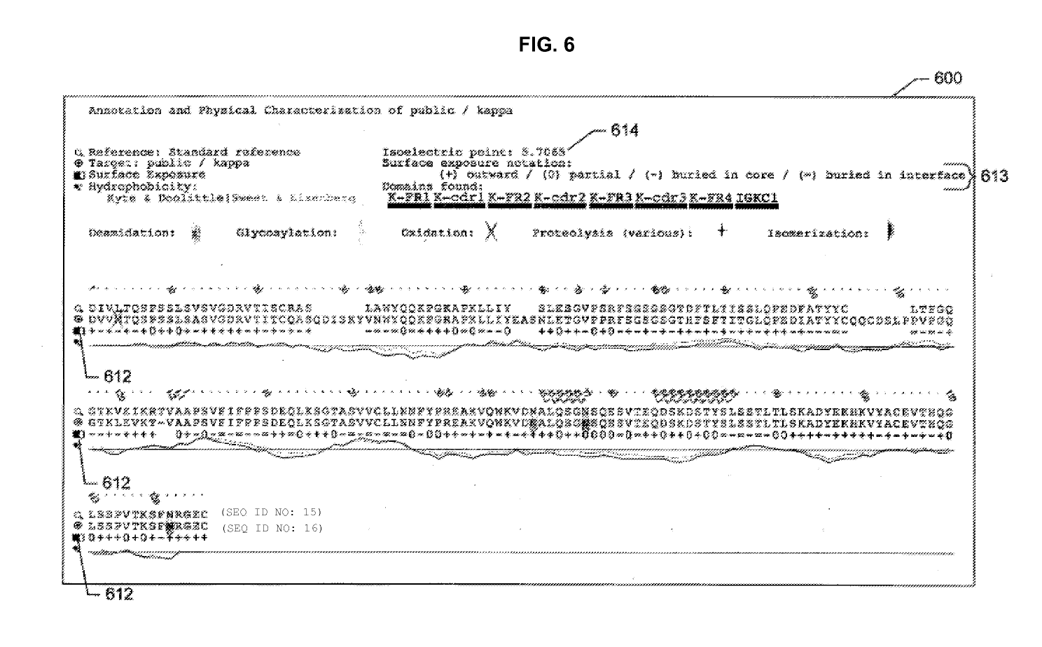

Once the computing device receives the alphabetic string indicative of the amino acid sequence, the computing device preferably displays an indication of surface exposure (block 504). For example, the computing device may display different symbols adjacent to the alphabetic string to indicate a level of surface exposure. In the example of FIG. 6, a surface exposure row 612 includes a symbol for each amino acid site. Each symbol is indicative of a level of surface accessibility of the represented amino acid position. As shown in key 613, in this example, a plus sign (e.g., “+”) indicates that the represented amino acid in that position is outward and therefore highly accessible to the solvent. A zero sign (e.g., “o”) indicates that the represented amino acid in that position is partially buried. A negative sign (e.g., “−”) indicates that the represented amino acid in that position is completely buried in a subunit hydrophobic core. An equal sign (e.g., “=”) indicates that the represented amino acid in that position is completely buried in a subunit interface. The determination of surface exposure may be determined using either (1) a static method, in which the outcome has been determined beforehand or (2) a dynamic method, in which the outcome is calculated on the fly each time.

Finally, the computing device calculates the isoelectric point 614 associated with the amino acid sequence based on the surface exposure and transfers the isoelectric point 614 to an output device such as a display (block 506). For example, the computing device may identify which amino acids in the amino acid sequence are near a surface of the antibody and which amino acids are not near the surface of the antibody (e.g., based on the data used to display the surface exposure row 612 generated by block 504). The isoelectric point 614 of the amino acid sequence may then be calculated using only the amino acids that are at and/or near a surface of the antibody (e.g., a surface pl). For example, the isoelectric point 614 may be calculated using just the amino acids associated with an outward exposure as indicated by the “+” symbol in the surface exposure row 612. Alternatively, the isoelectric point 614 may be calculated using just the amino acids associated with a partial exposure as indicated by the “o” symbol in the surface exposure row 612. In yet another example, the isoelectric point 614 may be calculated using just the amino acids associated with an outward exposure and a partial exposure as indicated respectively by the “+” symbol and the “o” symbol in the surface exposure row 612.

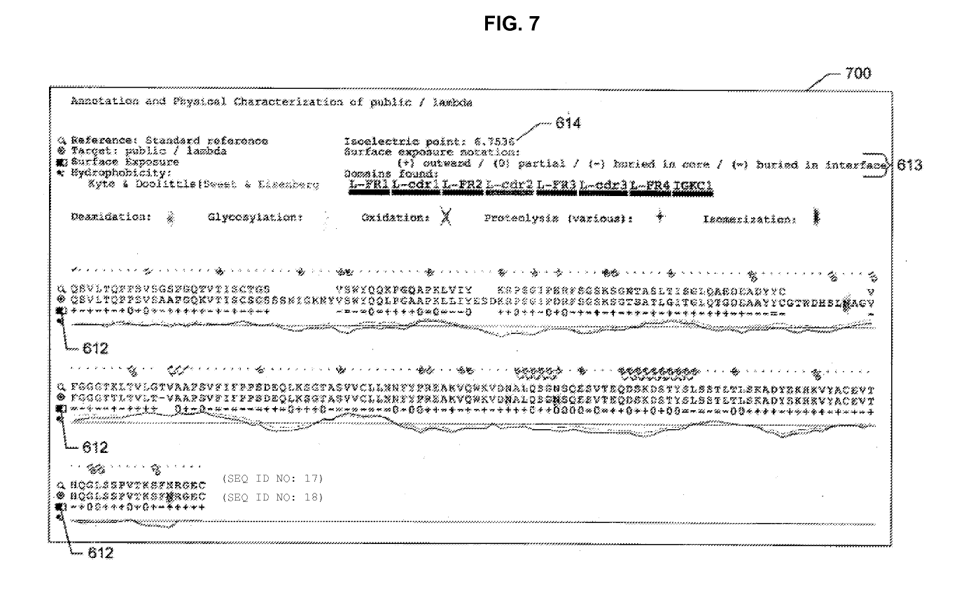

Another screen shot 700 of an example user interface for displaying alphabetic strings and associated chemical property predictions is shown in FIG. 7. Like the example of FIG. 6, the example of FIG. 7 includes a surface exposure row 612, which includes a symbol for each amino acid site indicative of a level of surface accessibility of the represented amino acid position. The example of FIG. 7 also includes an isoelectric point 614 associated with the amino acid sequence that may be based on the surface exposure.

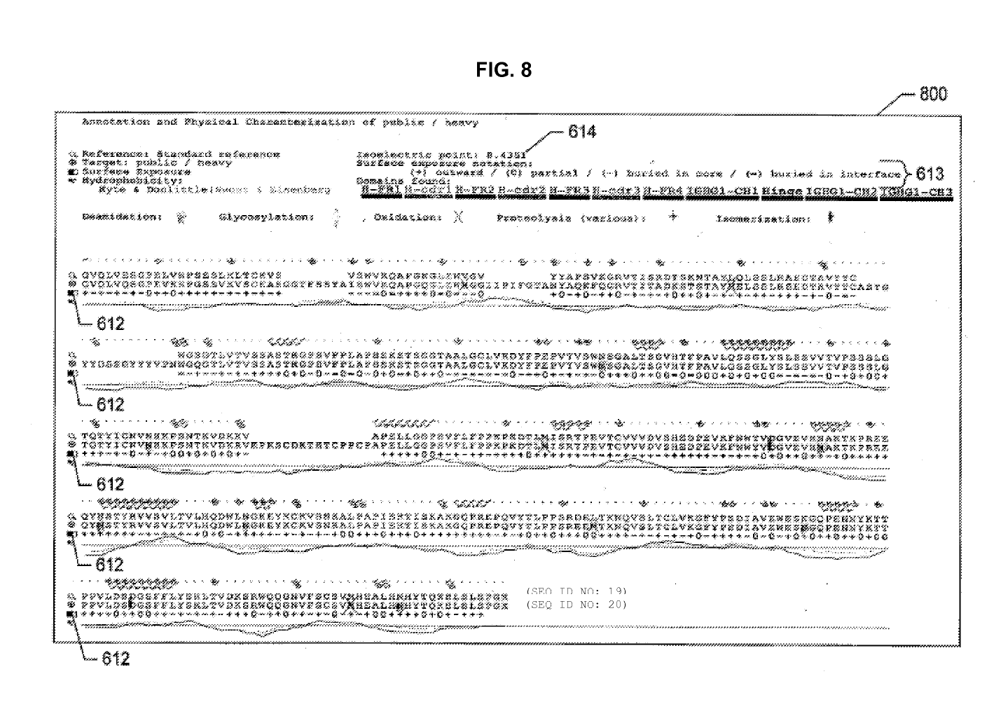

Yet another screen shot 800 of an example user interface for displaying alphabetic strings and associated chemical property predictions is shown in FIG. 8. Like the example of FIG. 6, the example of FIG. 8 includes a surface exposure row 612, which includes a symbol for each amino acid site indicative of a level of surface accessibility of the represented amino acid position. The example of FIG. 8 also includes an isoelectric point 614 associated with the amino acid sequence that may be based on the surface exposure.

### Protein Preparation and Formulation

The present disclosure provides methods and materials for determining an isoelectric point of a protein, which isoelectric point may be used for the preparation of a protein, including for purification (such as to select and utilize one or more buffers for purification of the protein) and/or for formulation. The methods may include one or more steps to purify the protein from a heterogeneous population of proteins and/or non-protein macromolecules (e.g., nucleic acids, endotoxin) and/or other contaminants. Such buffers may be used to stabilize a protein in solution.

A variety of methods are known in the art for purification of proteins, including, for example, purification of binding molecules such as antibodies and antibody fragments (see, e.g., Protein Purification: Principles, High-Resolution Methods, and Applications, 2nd Edition, 1997, Janson, J.-C., and Rydén. L. (Eds.), Wiley; Isolation and Purification of Proteins, 2003, Hatti-Kaul, R. and Mattiasson, B. (Eds.), CRC Press; Protein Purification Techniques: A Practical Approach, 2^{nd} Edition, 2001, Roe, S. (Ed.), Oxford University Press; Huse et al., 2002, J. Biochem. Biophys. Methods 51:217-231; Low et al., 2007, J. Chromatography 848:48-63; Hober et al., 2007, J. Chromatography 848:40-47; Aldington et al., 2007, J. Chromatography 848:64-78). Purification methods may include one or more chromatographic purification steps, wherein a purification step may involve one or more buffers. Chromatographic purification steps may include, for example, Protein A chromatography, ion exchange chromatography (e.g., cation exchange, anion exchange), hydrophobic interaction chromatography, ceramic hydroxyapetite chromatography, affinity chromatography and/or size exclusion chromatography. Proteins subjected to purification may be “crude” preparations of protein (e.g., microbial or mammalian cell culture supernatants, cell lysates) or partially purified preparations of protein previously subjected to one or more purification steps. Optionally, crude preparations of protein may be subjected to one or more steps of clarification to remove cell debris (e.g., centrifugation, filtration) concentration (e.g., tangential flow filtration), and or treatment with a nuclease (e.g., benzonase) to digest nucleic acids.

Ion exchange chromatography involves one or more buffers and separates compounds, such as proteins, based on the nature and degree of their ionic charge. In the case of proteins, ion exchange chromatography generally involves the binding of a protein to a charged matrix or resin under conditions where other protein or non-protein contaminants (e.g., nucleic acids, endotoxin) are not bound, followed by elution of the protein from the charges of the resin. The ion exchanger may comprise, a cationic exchanger, such as for example, a sulphopropyl cation exchanger, a carboxymethyl cation exchanger, a sulfonic acid exchanger, a methyl sulfonate cation exchanger, an SO₃-exchanger, or an ion exchanger such as for example, a DEAE, TMAE, and DMAE. Non-limiting examples of commercially available ion exchangers useful in the purification of proteins include DEAE-Sepharose Fast Flow, TSKgel SP-2SW, DEAE-Toyopearl 650S, TSKgel SuperQ-5PW, Q-Sepharose Fast Flow, TSKgel Q-STAT, Resource Q, TSKgel DNA-STAT, Mono Q, CM-Sepharose FF, TSKgel SP-STAT, CM-Toyopearl 650S, SP-Toyopearl 650S, S-Sepharose FF and the like. Protein A chromatography involves one or more buffers and involves the specific binding the Fc region of antibodies, but not most non-IgG contaminants, to immobilized protein A resin.

An important factor for binding of the protein in chromatographic purification steps such as Protein A and ion exchange chromatography is the pH of the buffer used to equilibrate and load the protein. Important factors for the elution are pH and/or ionic strength. Generally the selection of appropriate buffer conditions (e.g. pH) for use in purification will take into consideration the isoelectric point of the particular protein. Selection of a buffer pH that is the same as or very close to the isoelectric point of the protein may lead to undesirable aggregation or precipitation of the purified protein. Aggregation of proteins, including, for example, binding molecules such as antibodies and antibody fragments may be monitored, by a variety of methods, including as non-limiting examples by SEC-HPLC and/or light scattering measurement. In contrast, a buffer pH that is too different from the isoelectric point of the protein may not provide sufficient purification of the protein away from other protein or non-protein contaminants. Thus, it is important to accurately determine the isoelectric point of a protein in order to select and utilize a buffer for purification of the protein.

The present disclosure provide methods and materials for determining an isoelectric point of a protein, which isoelectric point may be used to select a pH for the preparation of a formulation of the protein. A variety of methods are known in the art for formulation of proteins, including, for example, where the proteins are binding molecules such as antibodies and antibody fragments (see, e.g., Protein formulation and delivery, 2^{nd} Edition, 2007, McNally, E. J., and Hastedt J. E. (Eds.), Drugs and the Pharmaceutical Sciences Series, Vol. 175, Taylor & Francis, Inc.; Carpenter et al., 2002, Pharm Biotechnol. 13:109-33; Patro et al., 2002, Biotechnol. Annu. Rev. 8:55-84; Forkjaer et al., 2005, Nat. Rev. Drug Discov. 4:298-306; Wang, 1999, Int. J. Pharma. 185:129-188). For example, for liquid formulations an isoelectric point of the protein as determined by the methods described herein may be used to select a pH for the formulation. The pH of the formulation may be selected to be above or below the isoelectric point of the protein, so as to stabilize the protein (e.g., decrease protein aggregation and/or increase protein solubility.

This disclosure is further illustrated by the following examples which are provided to facilitate the practice of the disclosed methods. These examples are not intended to limit the scope of the disclosure in any way.

### EXAMPLES

### Example 1

### Determination of an Isoelectric Point of a Protein

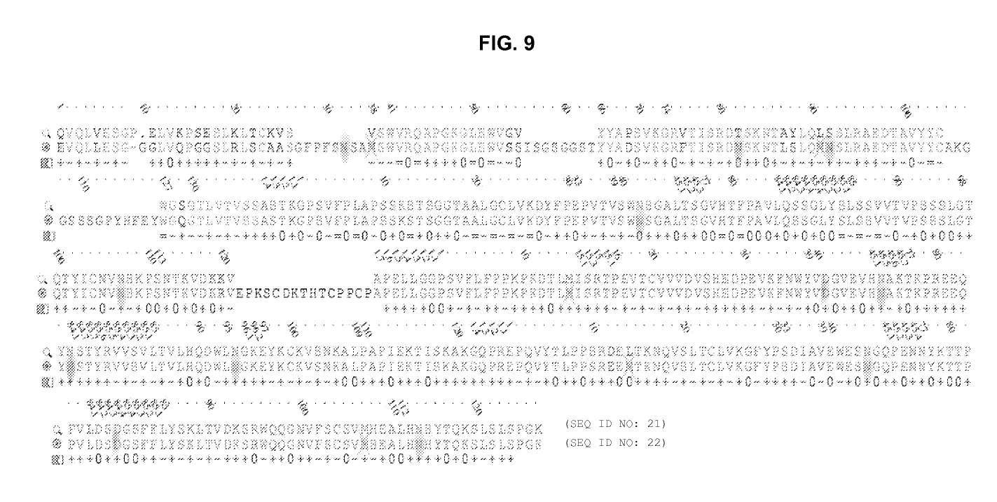

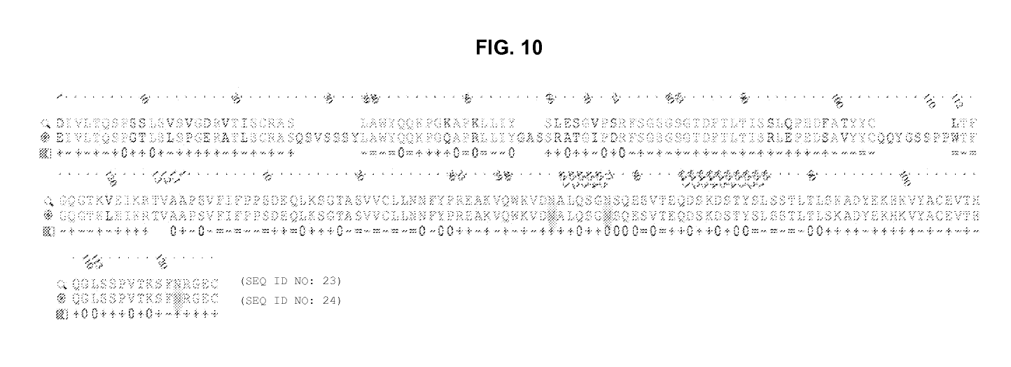

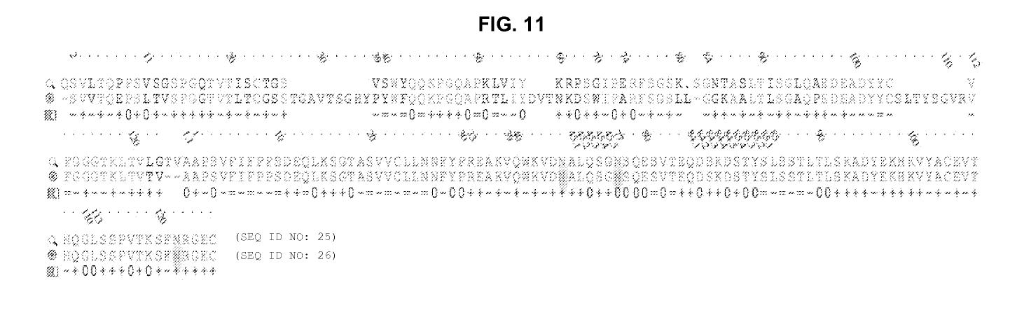

Calculated isoelectric points of proteins including, for example, an antibody may be determined as represented in FIGS. 9-11.

In an exemplary method, calculated isoelectric points of two exemplary antibodies, including a first antibody comprising a heavy chain (Genbank Accession No. CAC10540) and a kappa light chain (Genbank Accession No. BAC01559) and a second antibody comprising a heavy chain (Genbank Accession No. CAC10540) and a lambda light chain (Genbank Accession No. CAE18238) was determined. Each of the heavy, kappa and lambda chains (e.g., bottom string of amino acid residues in FIGS. 9-11, respectively) were aligned to a second sequence of amino acid residues (e.g., top string of amino acid residues in FIGS. 9-11, respectively) that are assigned a surface exposure notation (e.g., line consisting of +, o, − and = in FIGS. 9-11, respectively). These surface exposure notations included, for example, outward (+), partial (o), buried in core (−) or buried in interface (=). Amino acid residues in each of the two exemplary antibodies were then assigned the same surface exposure notation as assigned to the corresponding amino acid residue in the second sequence of amino acid residues. The pl of the two exemplary antibodies was then calculated using EMBOSS (e.g., the IEP module of EMBOSS) by taking into account amino acid residues including those classified as outward (+), partial (o), buried in core (−) or buried in interface (=) (e.g., may be referred to as the naïve pl). Additionally, the pl of the two exemplary antibodies was calculated by taking into account amino acid residues including those classified as outward (+) and partial (o) (e.g., may be referred to as surface pl). A naïve and a surface pl of the exemplary antibody comprising heavy chains and kappa light chains (e.g., Genbank Accession No. CAC10540 and BAC01559, respectively) were determined to be 8.1279 and 9.0996, respectively. A naïve and a surface pl of the exemplary antibody comprising heavy chains and lambda light chains (e.g., Genbank Accession No. CAC10540 and CAE18238, respectively) were determined to be 8.2309 and 9.4348, respectively.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method of preparing a protein, the method comprising:
a) identifying surface exposed amino acid residues in a sequence of amino acid residues of the protein;
b) assigning a pKa value to the surface exposed amino acid residues;
c) calculating the isoelectric point (pl) of the protein from the pKa values assigned to the surface exposed amino acid residues;
d) preparing the protein by at least one of: i) selecting a buffer with a pH not equal to the calculated isoelectric point of the protein and utilizing the selected buffer for purification of the protein; and ii) preparing a formulation of the protein with a pH not equal to the calculated isoelectric point of the protein.

2. The method of claim 1, wherein step d) comprises selecting a buffer with a pH not equal to the calculated isoelectric point of the protein and utilizing the selected buffer for purification of the protein.

3. The method of claim 2, wherein the protein is purified from a heterogeneous population of proteins.

4. The method of claim 1, wherein step d) comprises preparing a formulation of the protein with a pH not equal to the calculated isoelectric point of the protein.

5. The method of claim 4, wherein the method is a method for stabilizing a protein in solution.

6. The method of claim 2, further comprising preparing a formulation of the protein with a pH not equal to the calculated isoelectric point of the protein.

7. The method of claim 1, wherein the protein is an antibody or antibody fragment.

8. The method of claim 7, wherein the antibody or antibody fragment is an IgG, a Fab or a scFv.

9. The method of claim 1, wherein the pKa values are assigned to the surface exposed amino acid residues by the system of EMBOSS, DTASelect, Solomon, Sillero, Rodwell, Patrickios or Wikipedia

10. The method of claim 1, wherein all of the surface exposed amino acid residues are assigned a pKa value.

11. The method of claim 1, wherein the pl is calculated using the Henderson-Hasselbalch equation.

12. The method of claim 1, wherein the pl is calculated using the method of EMBOSS, DTASelect, Solomon, Sillero, Rodwell, Patrickios or Wikipedia.

13. The method of claim 1, wherein the surface exposed amino acid residues are identified as those amino acid residues with an ASA value equal to or greater than 2.

14. The method of claim 13, wherein the ASA values represent measured exposures for each amino acid residue.

15. The method of claim 13, wherein the ASA values represent estimated exposures for each amino acid residue.

16. The method of claim 7, wherein the surface exposed amino acid residues of the antibody are identified by aligning the sequence of amino acid residues of the antibody to a second antibody sequence of amino acid residues that are fixed to the “expo” line and are assigned an expo value as shown in FIGS. 1-4 and wherein amino acid residues of the antibody have expo values that are identical to corresponding positions in the second antibody.

17. The method of claim 16, wherein the surface exposed amino acid residues are exposed, outward oriented (+) amino acid residues.

18. The method of claim 16, wherein the surface exposed amino acid residues are partially exposed, surface oriented (o) amino acid residues.

19. The method of claim 16, wherein the surface exposed amino acid residues are exposed, outward oriented (+) amino acid residues and partially exposed, surface oriented (o) amino acid residues.

20. The method of claim 7, wherein the surface exposed amino acid residues of the antibody are identified by aligning the sequence of amino acids residues of the antibody to a second antibody sequence of amino acid residues that are fixed to an isoX line and are assigned an isoX value as shown in FIGS. 2-4 and wherein amino acid residues of the antibody have isoX values that are identical to corresponding positions in the second antibody.

21. The method of claim 20, wherein amino acid residues assigned an “IsoX” value of “•” are surface exposed amino acid residues.

22-111. (canceled)

112. A method for determining an isoelectric point of a protein, the method comprising:
a. receiving data indicative of a sequence of amino acid residues of the protein via an input device of a computing device;
b. identifying surface exposed amino acid residues in the sequence of amino acid residues;
c. assigning a pKa value to the surface exposed amino acid residues;
d. calculating the isoelectric point (pl) of the protein from the pKa values assigned to the surface exposed amino acid residues using the computing device; and
e. transferring the isoelectric point to an output device associated with the computing device.

113. A method for determining an isoelectric point of an antibody, the method comprising:
a. receiving data indicative of a sequence of amino acid residues of the antibody via an input device of a computing device;
b. identifying surface exposed amino acid residues in the sequence of amino acid residues by aligning the sequence of amino acids residues of the antibody to a second antibody sequence of amino acid residues that are fixed to an IsoX line and are assigned an isoX value as shown in FIGS. 2-4 and wherein amino acid residues of the antibody have isoX values that are identical to corresponding positions in the second antibody;
c. assigning a pKa value to the surface exposed amino acid residues;
d. calculating the isoelectric point (pl) of the protein from the pKa values assigned to the surface exposed amino acid residues using the computing device; and
e. transferring the isoelectric point to an output device associated with the computing device.

114-128. (canceled)
